(19) United States
(12) Patent Application Publication
(10) Pub. No.: US 2007/0299297 A1
(43) Pub. Date: Dec. 27, 2007

(54) Textured conforming shell for stabilization of the interface of precision heart assist device components to tissues

(76) Inventor: Robert Jarvik, New York, NY (US)

Correspondence Address: ROBERT JARVIK M.D.;c/o JARVIK HEART, INC., 333 W 52nd STREET, NEW YORK, NY, 10019, US

(21) Appl. No.: 11/474,818

(22) Filed: Jun. 26, 2006

(51) Int. Cl. A61N 1/362

(52) U.S. Cl. 600/16

(57) ABSTRACT

The blood contacting surfaces of heart assist devices must avoid excessive thrombus formation, which can break off and cause thromboembolism, become infected and cause other problems. Certain textured surface coatings, such as sintered titanium microsphere coatings, form a thin layer of living cells on the surface that becomes endothelized and is highly resistant to thrombus generation. Some of these coatings require high processing temperatures. Simple thick wall conduit tubes, which do not require high precision, coated with sintered microspheres, have been used successfully as inlet cannulae. Thick wall titanium pump components have also been successfully coated with sintered microspheres, using methods to retain their shape in the furnace and avoid excessive deformation. Blood pumps or portions of blood pumps that utilize high precision components subject to damage or warping if exposed to high temperatures cannot be directly coated. This applies to intraventricular and other blood pumps with precision heat sensitive components, such as polymer insulated wires, placed at least partly within an organ of the cardiovascular vascular system. The present invention provides a thin wall textured surface shell that is coated at high temperature and then, after finish machining, is affixed over the heat sensitive precision blood pump to serve as the interface with biological tissues.

### BACKGROUND OF THE INVENTION

In recent years ventricular assist devices used to support the pumping function of the natural heart have become increasingly successful. Long term patient survival in excess of five years has been achieved and many developers of ventricular assist devices are working on improved models intended to sustain patients in excess of a decade. Attachment of the blood pump is usually made by placement of an inlet cannula into the apex of the left ventricle. Alternatively, a cannula may be placed into the left atrium, right ventricle or right atrium. In some cases, blood pumps may be placed directly within the heart, such as the Jarvik 2000 device, U.S. Pat. No. 5,613,935, or some components of the device may be placed within the ventricle while other components are immediately adjacent to the heart, such as the hybrid flow pump of U.S. Pat. No. 5,824,070 or the rotary pump of U.S. Pat. No. 6,234,998 by Wampler.

It is important to establish a stable tissue junction between the cannula surface and the myocardial tissue in contact with it. It has long been known in the art that textured surfaces made of biocompatible materials can be engineered to form a thin layer of living tissue which becomes biologically continuous with the endocardial tissue at the cellular level. The material most commonly used for the device/tissue interface is a porous coating for the inlet cannula of a heart assist device that was developed by Thermoelectron Corporation, under a NIH funded research program during the 1980s. This material is comprised of a titanium alloy substrate having sintered titanium microspheres fused to the surface to form a porous coating having voids between the individual spheres into which cells grow and adhere. This material has been used to both coat the outer surface of the inlet cannula, as well as the inner surface of the cannula and the inner surface of the pusher plate pump housing in the HeartMate LVAD, the only heart assist device presently PMA approved by the FDA for permanent use. More than 2500 HeartMate devices having the porous microsphere coating have been implanted in patients with excellent results. The tissue coating on the cannula surface remains thin in a very high percentage of cases, without growing so thick as to restrict or occlude the inflow opening and without forming large thrombi on the surface.

Other types of porous surfaces have also been used on cannulae intended to remain in the heart long term, including polyester fabric used on the surface of apico-aortic conduits, or on the inlet cannulae of other heart assist devices such as the Novacor LVAD. Fabric is not as good as the porous microsphere surface if it is applied over an impermeable metal, because the fabric is thicker, and cells growing deep down within it, close to the metal it covers may not receive sufficient oxygen and nutrients from the blood and may not survive. Thus, if the layer of biological “neo-intema” on the fabric grows too thick, it may sluff off and cause an embolic stroke. The much higher rate of stroke which occurred with the Novacor heart compared to the HeartMate, was attributed to problems with the surface of the inlet cannula. Recently, the Novacor inlet cannula was changed to Goretex (microporous PTFE) with improved results.

The use of a particular porous coating on or within an implanted blood pump may or may not succeed, depending on the flow conditions at the porous surface, as well as the characteristics of the surface itself. For example, the HeartMate II blood pump is a miniature axial flow pump which was initially fabricated with the same sintered microsphere coating as used with the HeartMate I, on the stationary inner surfaces of the pump. The spinning rotor was not coated with sintered microspheres. This design failed, because under the higher shear and turbulence within the axial pump, thick layers of thrombus adhered to the porous surface which occluded the pump. The design was then changed, using smooth polished titanium surfaces throughout the HeartMate II, and the pump occlusion problem was solved. The HeartMate II now uses an inlet cannula virtually identical to the HeartMate I inlet cannula, coated inside and out with sintered titanium microspheres.

The Jarvik 2000 heart does not use an inlet cannula, because it is placed directly into the heart, with the pump and motor actually within the left ventricle. Until the introduction of the present invention, the Jarvik 2000 used a smooth outside surface. This surface does not form a cellular connection to the myocardium, at lease not in the first few weeks postoperative. There is thus an open crevice between the outside of the Jarvik 2000 housing, and the myocardial tissue where the pump is implanted across the left ventricular wall. Initially, thrombus may form in this crevice and occasionally has broken free to cause a thrombo-embolic stroke. To one skilled in the art, use of a porous coating on the outside of the Jarvik 2000, would appear an obvious solution in creating adherent tissue interface between the pump and the natural heart tissue by applying the titanium microsphere coating to the outside of the device. But although this might appear obvious in concept, its implementation has many drawbacks. Fusing the titanium microspheres to the surface is a high temperature process which requires temperatures above 1,000 degrees Celsius. This cannot be done after the motor stator is mounted within the pump housing, since the insulating materials on the motor wire would be destroyed. Other heat sensitive materials, such as magnets which might be used in other VAD designs, must not be installed in the parts prior to coating with microspheres. If the parts which must be coated have a thin wall section, they will distort due to stresses which occur from the high temperatures. Precision of size, roundness, and straightness will be lost. If the coating is done first, and then the final machining is done, even if the parts are annealed, they will be subject to warping from internal stress. The machining properties of Ti6A1V4, the preferred alloy, are also unfavorably altered by the high temperature sintering process. Additionally, the final porous surface of the microspheres must be completely clean. This makes machining the coated parts problematic, because microscopic particles in the coolant or from other sources will lodge in the pores of the surface and contaminate it.

The present invention provides a solution to all of these problems by providing a porous microsphere coated “shell” which is manufactured separate from the blood pump or inlet cannula components of the blood pump, and is affixed at a final or near final stage of assembly of the ventricular assist device, in a clean environment. The porous surfaced “shell” may be a paper thin covering conforming to the surface shape of the part of the pump or cannula it covers, such as a cylinder, cone, dome, or irregular form. It may be attached by one of numerous methods, including the use of screws or other fasteners, press fitting, crimping, shrink fitting, bonding, welding, or other methods.

The completed heart assist device thus has a textured coating affixed over the surface of a precision assembly.

### OBJECTS OF THE INVENTION

It is an object of the present invention to minimize the amount of thrombus which forms at the interface between the parts of a cardiac assist device implanted into or upon the natural heart and the living tissue on the endocardial surface of the heart in contact with the device.

It is another object of the invention to provide a thin-walled conforming textured covering which forms an interface for tissue adhesion between the living tissue of the natural heart, and the portion of a precision heart assist device in contact with the natural heart.

Another object of the invention is to provide a textured structure which is produced by high temperature processing separate from the low temperature processing of precision components of the blood pump and inflow structure.

It is a further object of the invention to provide a covering for precision cardiac assist devices, comprised of a sintered titanium microsphere surface layer that has microsphere metallurgy, microsphere size, porosity, thickness, and cleanliness equivalent to surfaces successfully used on non-precision heart assist components.

It is a still further object of the invention to provide a precision heart assist blood pump which heals to the natural heart tissue in a safe stable manner without the need for anticoagulant medication to prevent thrombus formation at the junction of the device to the heart.

### THE DRAWINGS

FIG. 1 is a schematic longitudinal section of a prior art device, the Jarvik 2000 heart, implanted into the apex of the left ventricle.

FIG. 2 is a schematic illustration of a prior art hybrid blood pump implanted into the apex of the left ventricle.

FIG. 3 is a schematic illustration of a prior art partially magnetically suspended blood pump implanted into the apex of the left ventricle.

FIG. 4 is a schematic longitudinal section of a prior art device, having a magnetically and hydrodynamically supported rotor, which is implanted at the apex of the left ventricle in essentially the same way as the pump illustrated in FIG. 2.

FIG. 5 is an illustration of a Jarvik 2000 heart with a smooth outer surface showing the formation of thick thrombus surrounding it.

FIG. 6 is a longitudinal section of the wall structure and coating of the preferred embodiment of the present invention also shown in FIG. 8.

FIG. 7 is an enlarged detail of a portion of the device of FIG. 6.

FIG. 8 is an illustration of the device of the present invention fitted in place over the housing of an intraventricular blood pump.

FIG. 9 is an illustration of the textured conforming shell affixed onto a Jarvik 2000 heart and implanted into the apex of the heart.

FIG. 10 is a sectional view of the blank which is used to fabricate preferred embodiment of the present invention showing the material that is removed by machining and the material remaining which comprises the wall of the finished shell.

FIG. 11 is an enlarged longitudinal section of a portion of the end of the blank of FIG. 10, after coating with microspheres.

FIG. 11A is a section of a portion of the finished textured conforming shell after final machining.

FIG. 12 is an illustration showing the material of the blank that is removed by machining to obtain the finished textured shell.

### SPECIFIC DESCRIPTION OF THE INVENTION

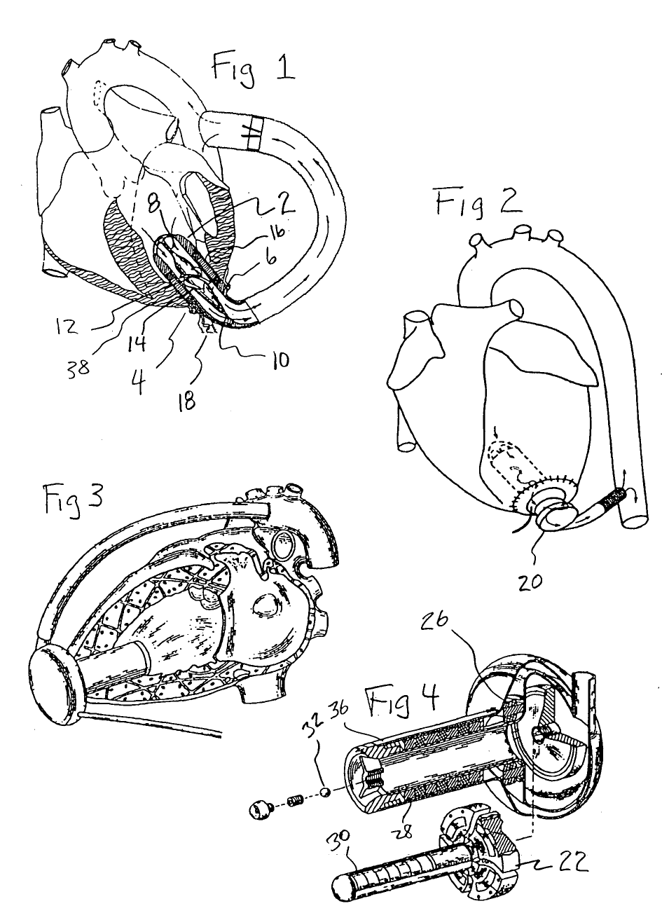

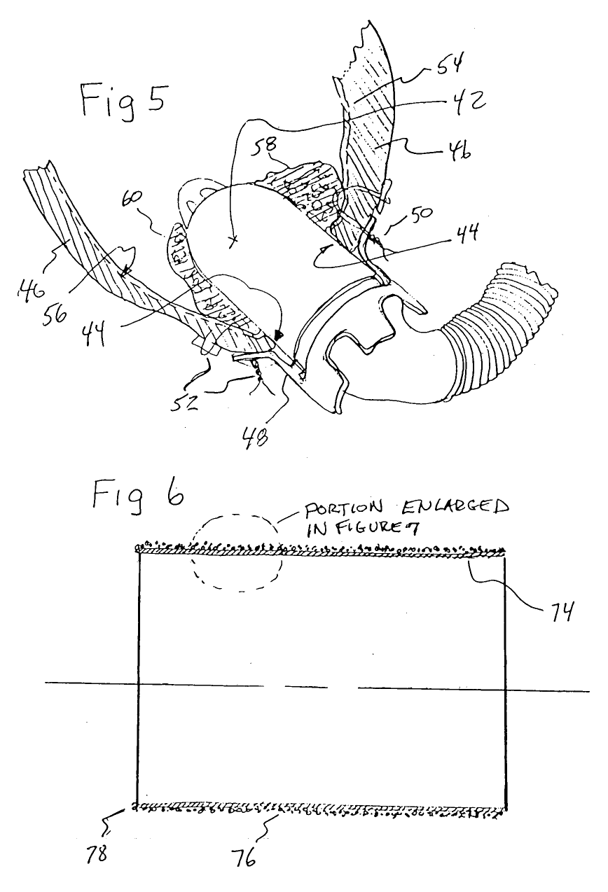

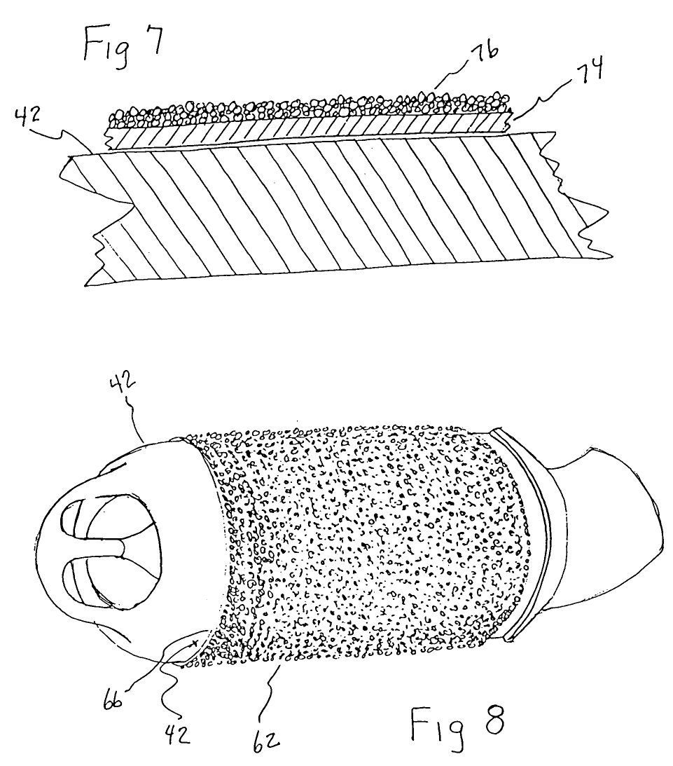

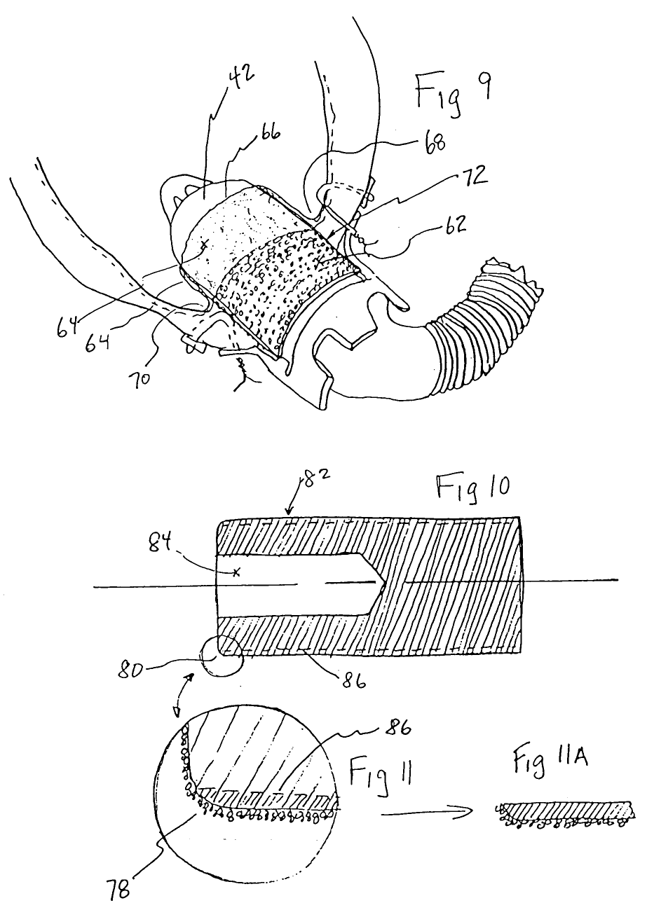

The present invention provides a thin wall “shell” which surrounds a ventricular assist device implanted in the heart. The assist device may be implanted into any of the four chambers of the natural heart. The most common position used in present clinical practice is the left ventricle, as illustrated in FIG. 1, from the prior art. An intraventricular axial flow pump 2 is positioned in the left ventricle and is implanted through a hole in the wall of the left ventricle 4, typically cut with a special instrument called a coring knife. The axial pump includes a rotor 6, supported on bearings 8, 10. The rotor supports impeller blades 12, 14. The armature of a motor, 16, receives electric power via a cable, 18. Properly timed power pulses induce magnetic fields in the motor armature which apply magnetic force to magnets within the rotor. The blood pumping device may be substantially implanted inside the heart, as in the prior art embodiment of FIG. 1, or it may utilize some elements within the heart and some outside the heart, as illustrated in the prior art invention of FIGS. 2 and 4, where part of the bearing and rotor structure is located within the heart, and part of the pump structure, such as a centrifugal diffuser 20, or a centrifigual pump impeller 22, is located just outside the natural heart.

Referring to the prior art inventions shown in FIGS. 1-4, note that in each case the structure within the ventricular chamber and the structure within the ventricular wall contains precision heat sensitive components, such as motor windings 26, magnets 28, 30, bearing components 8, 10, 32 and thin walled metal housing components 36, 38, which would be subject to warping if placed in a very hot furnace.

Blood pumps of this general configuration can be manufactured to high precision using standard machining methods with proper coolant. FIG. 5 illustrates a problem which may occur in some patients with devices having a smooth highly polished surface. The intraventricular housing of an axial blood pump 42, is in contact with the cut wall of the ventricle 46, at position 44, which represents the circumference of a hole cut through the ventricle, into which the pump has been inserted, and affixed via sewing cuff 48, using sutures 50, 52. The inner lining of the heart, called the endocardium, is generally indicated by dotted lines 54, 56. A large circumferential thrombus, 58, 60, surrounds the blood pump housing 42. Typically, this type of thrombus can first form in the crevice at the cut junction of the myocardium 44, where the freshly cut heart tissue acts as a stimulus to clotting, due to release of substances known as tissue thromboplastins. After initial formation of the thrombus in the crevice it may enlarge to become a large thrombus, as shown at 58, 60. Motion of the heart causes motion of the device relative to the thrombus which stimulates it to enlarge.

FIG. 6 illustrates the device of the present invention, in a cylindrical configuration. The thin wall “shell” 74 is a simple tube coated with sintered titanium microspheres, 76. This is better seen in the enlarged illustration FIG. 7, where the wall of blood pump housing, 42, has also been shown in longitudinal section. At the end of the textured shell that makes the junction to the smooth surface of the blood pump housing 66, the thin wall of the shell has a radius on the edge which is coated with microspheres. This is better seen in FIG. 11 where the radius 78 on the end of the tube is enlarged. Refering to FIG. 8, the textured microsphere coating 62, extends directly against the smooth polished surface 42 of the blood pump housing.

FIG. 9 illustrates a blood pump surrounded by a textured “shell” 62 after implantation into a heart and formation of a layer of neo-intima 64, over the textured portion of the “shell”. This neo-intima contains cells which grow into the spaces within the textured surface, and attach a surface layer of living tissue firmly to the device. The neo-intima makes contact with the endocardium at the cellular level and this living lining develops an endothelial cell surface which prevents blood clots from forming and growing to a large mass as shown in FIG. 5, at 58, 60. In the preferred embodiment, the textured shell does not cover the entire outer surface of the device, but ends at a position generally indicated at 66, in FIGS. 8 and 9. The exposed end of the blood pump housing 42, as seen in FIG. 8, is a smooth highly polished surface. The neointemal tissue which grows into the textured surface covers only the textured surface. It stops at the junction of the textured to the smooth surface 66.

The portion of the myocardium in contact with the textured surface 72, in FIG. 9, forms connective tissue in growth into the spaces within the surface that further helps anchor the device to the natural heart tissue.

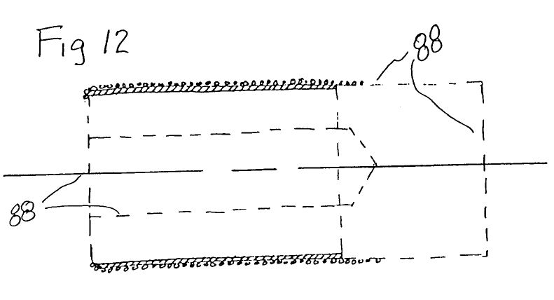

FIG. 10 illustrates the method of manufacture of the preferred embodiment of the invention. A titanium alloy blank, 82, is machined with an outer diameter approximately 0.010-0.015″ greater than the outer diameter of the blood pump housing, over which it will be fitted. A pilot hole 84 may be used in the blank to facilitate machining after coating and sintering of the microspheres. The edge of the blank has a machined radius, approximately equal to the wall thickness of the completed “shell”. The dotted line 86, in FIGS. 10 & 11 indicates the final amount of material which will comprise the “shell” thickness after finish machining. FIG. 11A shows a longitudinal section of the finished shell. FIG. 12 shows the portion of the blank (in dotted lines 88) which must be removed by machining to obtain the finished device.

Typically, the blank has a thick wall section or is solid, so when this is placed in the furnace at high temperature to sinter the microspheres, negligible warping occurs. The final machining is done with a very clean filtered coolant, so that the pores between the microspheres remain extremely clean. The machining process is also carefully controlled to prevent heating sufficient to warp the finished part. Even if slight warping occurs, when the shell is placed over the pump housing, as seen in FIG. 8, it conforms to the cylindrical shape of the blood pump housing. The textured “shell” is manufactured with a tight slip fit if it is to be welded to the pump housing. This may be accomplished without damage to the pump motor inside the pump housing, by heat sinking, and by using small spot laser welds. By making the surface coating on the blank (82) relatively thick, such as 0.015-0.025″ thick, it becomes strong enough that the machining process may remove the entire blank in which case the entire “textured shell” is comprised only of sintered microspheres.

The structure of the present invention described above is feasible to manufacture by the method described, and when affixed to the surface of a ventricular assist device, by welding or by another suitable method, produces a blood pump substantially similar to the device that could be obtained if a very low temperature sintering method existed, which allowed the finished precision blood pump to be coated directly. Since no sufficiently low temperature sintering process exists, it can be seen that the present invention discloses an original and important structure to improve the performance of mechanical cardiac assist devices.

The information disclosed in the description of the present invention is intended to be representative of the principles I have described. It will thus be seen that the objects of the invention set forth above and those made apparent from the preceding description are efficiently obtained and that certain changes may be made in the above articles and constructions without departing from the scope of the invention. It is intended that all matter contained in the above description and shown in the accompanying drawings shall be interpreted as illustrative but not in a limiting sense. It is also understood that the following claims are intended to cover all of the generic and specific features of the invention herein described and all statements of the scope of the invention which, as a matter of language, might be said to fall there between.

I claim:

1. A surface textured metal shell adapted to be affixed onto or surrounding a ventricular assist device, said assist device comprised of components including heat sensitive precision components that would be damaged if exposed to the minimum temperatures required to produce said coating on said shell.

2. The surface textured metal shell of claim 1 in which the textured surface is comprised of sintered titanium or sintered titanium alloy microspheres.

3. The surface textured metal shell of claim 1 having so thin a wall thickness that it relies upon the structural strength of the assist device components around which it is affixed, to prevent distortion of its shape in use.

4. The surface textured metal shell of claim 1 in which a sintered microsphere layer is adherent to a thin shell-like substrate layer, the thickness of the microsphere layer being approximately equal or greater than the thickness of the substrate layer.

5. The surface textured metal shell of claim 1 in which a sintered microsphere layer is adherent to a thin shell-like substrate layer, the thickness of the substrate layer being approximately 0.003-0.008″ thick and approximately equal or less than the thickness of the microsphere layer.

6. The surface textured metal shell of claim 1 having a non-porous substrate wall upon which a porous surface layer has been bonded, said substrate wall thickness equal to or less than 3 times the thickness of the porous surface layer adherent thereupon.

7. The method of fabrication of a surface textured metal shell, for application onto a ventricular assist device, comprised of:
a. processing a blank having sufficient thickness and strength to withstand high enough temperature for the coating process without excessive deformation, to produce the required coating, and
b. following high temperature coating, machining away most or all of the blank material to leave a thin walled shell structure having the desired textured surface.

8. A surface textured metal shell adapted to form an interface between the intra-myocardial, intra-ventricular, or intra-atrial components of a ventricular assist device and the adjacent myocardial tissue and blood, comprised of a sintered microsphere coated metal shell, affixed surrounding motor, bearing, or magnetic components of said assist device that would be damaged if exposed to high enough temperatures to create a sintered metal microsphere coating during processing.

9. A surface textured metal shell adapted to be fixed onto a ventricular assist device and form an interface between the intra-myocardial, intra-ventricular, or intra-atrial components said device and the adjacent myocardial tissue and blood, comprised solely of a thin walled porous material comprised of sintered titanium or titanium alloy microspheres adherent to each other by means of their sintered contact points.
